(19) United States
(12) Patent Application Publication
(10) Pub. No.: US 2005/0228484 A1
(43) Pub. Date: Oct. 13, 2005

(54) Modular endovascular graft

(75) Inventors: W. Patrick Stephens, Santa Rosa, CA (US); Jenine S. Vinluan, Santa Rosa, CA (US); Issac J. Zacharias, Santa Rosa, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TriVascular, Inc., Santa Rosa, CA (US)

(21) Appl. No.: 11/077,938

(22) Filed: Mar. 11, 2005

Related U.S. Application Data
(60) Provisional application No. 60/552,132, filed on Mar. 11, 2004.

(51) Int. Cl. A61F 2/06

(52) U.S. Cl. 623/1.16; 623/1.21; 623/1.35; 623/1.36

(57) ABSTRACT

A modular endovascular graft wherein the graft body sections may be secured to each other by a variety of methods, including attachment elements having inflatable circumferential channels that interlock with other inflatable channels, recessed pockets or the like. Embodiments may have inflatable cuffs for sealing against an inside surface of a patient's fluid flow lumen, such as a blood vessel. Embodiments may also include expandable stents secured to and extending from ends of the various graft body sections for mechanically securing the graft, or sections thereof, to the patient's fluid vessels.

### CROSS-REFERENCE TO RELATED APPLICATIONS

The present application claims benefit to U.S. Provisional Application Ser. No. 60/552,132 entitled “Modular Endovascular Graft,” filed Mar. 11, 2004, the complete disclosure of which is incorporated herein by reference.

### BACKGROUND OF THE INVENTION

An aneurysm is a medical condition indicated generally by an expansion and weakening of the wall of an artery of a patient. Aneurysms can develop at various sites within a patient's body. Thoracic aortic aneurysms (TAAs) or abdominal aortic aneurysms (AAAs) are manifested by an expansion and weakening of the aorta, and are serious and life threatening conditions for which intervention is generally indicated. Existing methods of treating aortic aneurysms include invasive surgical procedures with graft replacement of the affected vessel or body lumen or reinforcement of the vessel with a graft.

Surgical procedures to treat aortic aneurysms tend to have relatively high morbidity and mortality rates due to the risk factors inherent to surgical repair of this disease. Painful recoveries involving long hospital stays are typical as well. This is especially true for surgical repair of TAAs, which is generally regarded as involving higher risk and more difficulty when compared to surgical repair of AAAs. An example of a surgical procedure involving repair of an aortic aneurysm is described in a book titled “Surgical Treatment of Aortic Aneurysms” by Denton A. Cooley, M.D., published in 1986 by W.B. Saunders Company.

Due to the inherent risks and complexities of surgical repair of aortic aneurysms, endovascular repair has become a widely-used alternative therapy, most notably in treating AAAs. Early work in this field directed towards percutaneous endovascular therapy is exemplified by Lawrence, Jr. et al. in “Percutaneous Endovascular Graft: Experimental Evaluation”, Radiology (May 1987) and by Mirich et al. in “Percutaneously Placed Endovascular Grafts for Aortic Aneurysms: Feasibility Study,” Radiology (March 1989).

Commercially available endoprostheses for the endovascular treatment of AAAs include the AneuRx™ stent graft manufactured by Medtronic, Inc. of Minneapolis, Minn., the Zenith™ stent graft system sold by Cook, Inc. of Bloomington, Ind., the PowerLink® stent-graft system manufactured by Endologix, Inc. of Irvine, Calif., and the Excluder® stent graft system manufactured by W.L. Gore & Associates, Inc. of Newark, Del. A commercially available stent graft for the treatment of TAAs is the TAG™ system manufactured by W.L. Gore & Associates, Inc.

When deploying such devices by catheter or other suitable instrument, it is advantageous to have a flexible and low profile stent graft and delivery system, particularly for patients with small vessels and/or tortuous vascular anatomies. Many of the existing devices for the endovascular treatment of aortic aneurysms, while representing significant technological advancements over previous devices, remain relatively large in transverse profile, often up to 24 French. In addition, some existing systems have greater than desired longitudinal stiffness, which can complicate the delivery process. As such, relatively non-invasive, even percutaneous, endovascular treatment of aortic aneurysms is not available for many patients that would benefit from such a procedure and can be more difficult to carry out for those patients for whom the procedure is indicated. What has been needed is a graft that can be safely and reliably deployed using a flexible low profile system.

### BRIEF SUMMARY OF THE INVENTION

Advantages in the treatment of fluid flow vessels of a patient's body such as ease of deployment and low profile delivery can be achieved by use of a modular endovascular graft design. In addition, advantages may be achieved by the use of modular inflatable grafts or stent grafts that include inflatable channels or cuffs, and in some embodiments, a network of inflatable channels that provide mechanical support and rigidity for the graft. Inflatable channels or cuffs may also be useful for providing a seal against an inside surface of a patient's fluid vessel and when used in combination with expandable stents which are axially separated or distinct from the cuffs or channels. The sealing function of the cuffs or channels may be separated from an anchoring or securing function of an expandable stent.

In one embodiment, the present invention provides a modular endovascular graft. The graft comprises a first graft body section that is at least partially inflatable. A second graft body section is securable to at least a portion of the first graft body section. In one configuration, both the first graft body section and the second graft body section are at least partially inflatable.

In a further embodiment, a modular endovascular graft has a first graft body section with a first fluid flow lumen bounded by a first wall portion. A first attachment element is disposed on the first wall portion and an inflatable cuff surrounds the first fluid flow lumen and extends radially from the first wall portion when in an inflated state. A second graft body section has a second fluid flow lumen bounded by a second wall portion. A second attachment element is disposed on the second wall portion which is configured to be secured to the first attachment element with the first fluid flow lumen sealed to the second fluid flow lumen.

In another embodiment, a modular endovascular graft has a first graft body section with a first fluid flow lumen bounded by a first wall portion and a first attachment element that includes a first inflatable element disposed on the first wall portion. A second graft body section has a second fluid flow lumen bounded by a second wall portion and a second attachment element disposed on the second wall portion which is configured to engage the first inflatable element when the first inflatable element is in an inflated state to prevent axial separation of the first and second graft body sections.

In another embodiment, a modular endovascular graft includes a first graft body section having a first fluid flow lumen and a first inflatable element that has a first reduced circumference shoulder portion on an inner surface of the first graft body section when the element is in an inflated state. A second graft body section has a second fluid flow lumen and is secured to the first graft body section by a second reduced circumference shoulder portion that mechanically engages the first reduced circumference shoulder portion to prevent axial separation of the first and second graft body sections.

In another embodiment, a bifurcated modular endovascular graft includes a main graft body section with a main fluid flow lumen therein, an ipsilateral port in fluid communication with the main fluid flow lumen and a contralateral port in fluid communication with the main fluid flow lumen. An ipsilateral attachment element is disposed on the main graft body section adjacent the ipsilateral port. A contralateral attachment element disposed on the main graft body section adjacent the contralateral port. An ipsilateral graft body section having an ipsilateral fluid flow lumen therein and a first attachment element disposed adjacent a proximal end of the ipsilateral graft body section is secured to the ipsilateral attachment element with the ipsilateral fluid flow lumen sealed to the main fluid flow lumen. A contralateral graft body section having a contralateral fluid flow lumen and a second attachment element disposed adjacent a proximal end of the contralateral graft body section is secured to the contralateral attachment element with the contralateral fluid flow lumen sealed to the main fluid flow lumen.

In yet another embodiment, a modular endovascular graft includes a first graft body section having a first fluid flow lumen bounded by a first wall portion, a first attachment element disposed on an outside surface of the first wall portion and a radial compression member secured to and disposed about the first graft body section at least partially over the first attachment element. The modular endovascular graft also includes a second graft body section having a second fluid flow lumen bounded by a second wall portion, a second attachment element disposed on an inside surface of the second wall portion engaged with the first attachment element with the first fluid flow lumen sealed to the second fluid flow lumen. The radial compression member applies inward radial force to the joint between the first attachment element and the second attachment element in order to enhance the strength of the joint.

In an embodiment of a method of treating a fluid flow vessel of a patient, a modular endovascular graft is provided including a first graft body section having a first fluid flow lumen and a first inflatable element that comprises a first reduced circumference shoulder portion on an inner surface of the first graft body section when the element is in an inflated state. The modular endovascular graft also includes a second graft body section having a second fluid flow lumen and is secured to the first graft body section by a second reduced circumference shoulder portion that mechanically engages the first reduced circumference shoulder portion to prevent axial separation of the first and second graft body sections. The first graft body section is deployed within a desired location of the patient's fluid flow vessel. The second graft body section is deployed adjacent the first graft body section such that the second attachment element is adjacent the first inflatable element. The first inflatable element is then inflated so as to engage the second attachment element and secure the first graft body section to the second graft body section.

These and other advantages of embodiments of the invention will become more apparent from the following detailed description of the invention when taken in conjunction with the accompanying exemplary drawings.

### BRIEF DESCRIPTION OF THE DRAWINGS

FIG. 1 shows an elevational view of a bifurcated modular endovascular graft having ipsilateral and contralateral graft body sections secured to a main graft body section.

FIG. 1A is a transverse cross sectional view of the bifurcated modular endovascular graft of FIG. 1 taken along lines 1A-1A of FIG. 1.

FIG. 1B is a transverse cross sectional view of the bifurcated modular endovascular graft of FIG. 1 taken along lines 1B-1B of FIG. 1.

FIG. 2 is an elevational view in longitudinal section of the graft of FIG. 1.

FIG. 2A illustrates the graft of FIG. 2 deployed within an abdominal aortic aneurysm.

FIG. 3 is an enlarged view of the encircled portion 3-3 of the modular endovascular graft of FIG. 2 showing the joint between the ipsilateral graft body section and the main graft body section.

FIG. 3A is an enlarged view of the encircled portion 3-3 of the modular endovascular graft of FIG. 2 showing the joint between the ipsilateral graft body section and the main graft body section wherein the ipsilateral graft body section is displaced distally illustrating an adjustable length feature of the joint.

FIG. 4 illustrates an alternative embodiment of the joint between the ipsilateral graft body section and the main graft body section shown in FIG. 3.

FIG. 5 illustrates an alternative embodiment of the joint between the ipsilateral graft body section and the main graft body section shown in FIG. 3.

FIG. 6 illustrates the joint between the ipsilateral graft body section and the main graft body section of FIG. 5 with the ipsilateral graft body section displaced distally and engaging a different combination of attachment elements illustrating the adjustable length feature of the embodiment.

FIG. 7 illustrates an exploded view in partial section of an ipsilateral graft body section having a radially enlarged axial section with a reduced circumference shoulder portion configured to engage a recessed pocket of a main graft body section.

FIG. 8 illustrates the enlarged axial section of the ipsilateral graft body section engaged in the recessed pocket of the main graft body section.

FIG. 9 illustrates an alternative embodiment of the joint between the ipsilateral graft body section and the main graft body section shown in FIG. 3 wherein a first attachment element is engaged with and secured to a second attachment element.

FIG. 9A is a transverse cross section of the joint of FIG. 9 taken along lines 9A-9A of FIG. 9.

FIG. 10 illustrates an embodiment of a first attachment element for the joint of FIG. 9 wherein the first attachment element includes a plurality of resilient loops.

FIG. 11 illustrates an embodiment of a second attachment element for the joint of FIG. 9 wherein the second attachment element includes a plurality of resilient hooks configured to engage the resilient loops of FIG. 10.

FIG. 12 illustrates an embodiment of a first attachment element for the joint of FIG. 9 wherein the first attachment element includes a plurality of resilient pins.

FIG. 13 illustrates an embodiment of a second attachment element for the joint of FIG. 9 wherein the second attachment element includes a mesh having a plurality of apertures configured to engage the pins of FIG. 12 when the first and second attachment elements are pressed together.

FIG. 14 illustrates an embodiment of a first attachment element for the joint of FIG. 9 wherein the first attachment element includes a plurality of resilient buttons having an enlarged head portion disposed through apertures of the second attachment element which is a mesh having a plurality of apertures configured to allow entry of the buttons of FIG. 14 when the first and second attachment elements are pressed together with the mesh in a circumferentially restrained state and wherein the mesh captures the enlarged head portion of the buttons when the mesh is in a circumferentially expanded state.

FIG. 15 illustrates the enlarged head portion of the resilient buttons of FIG. 14 captured by the apertures of the mesh that is in a circumferentially expanded state.

FIG. 16 illustrates an ipsilateral attachment element disposed near an ipsilateral port of a main graft body section with a radial compression member disposed substantially over the ipsilateral attachment element.

FIG. 17 illustrates a proximal end portion of an ipsilateral graft body section having a first attachment element disposed on an inside surface of the ipsilateral graft body section and an inflatable cuff disposed near the proximal end of the ipsilateral graft body section.

FIG. 18 illustrates a sandwiched joint between the main graft body section and the ipsilateral graft body section wherein the ipsilateral attachment element is engaged with and secured to the first attachment element and the junction between the attachment elements is being compressed by the inflatable cuff in an inflated state which is further compressed by the radial compression member disposed about the inflatable cuff.

FIG. 19 illustrates a perspective view of the joint of FIG. 18 where the molding of the inflatable cuff about the elongate elements of the radial compression member may be seen which further secures the joint between the main graft body section and the ipsilateral graft body section.

FIG. 20 is an elevational view in partial section of an alternative embodiment of attachment elements of graft sections wherein protuberances disposed on an expandable cylindrical member are configured to engage the openings of a mesh or similar structure.

FIG. 21 is an enlarged view of an embodiment of a mesh structure for the attachment element embodiment of FIG. 20.

FIG. 22 illustrates a joint between the attachment elements of the graft sections of FIG. 20.

FIGS. 23 and 24 illustrate an alternative embodiment of the joint between the ipsilateral graft body section and the main graft body section shown in FIG. 3 wherein a first attachment element is securable to a second attachment element.

### DETAILED DESCRIPTION

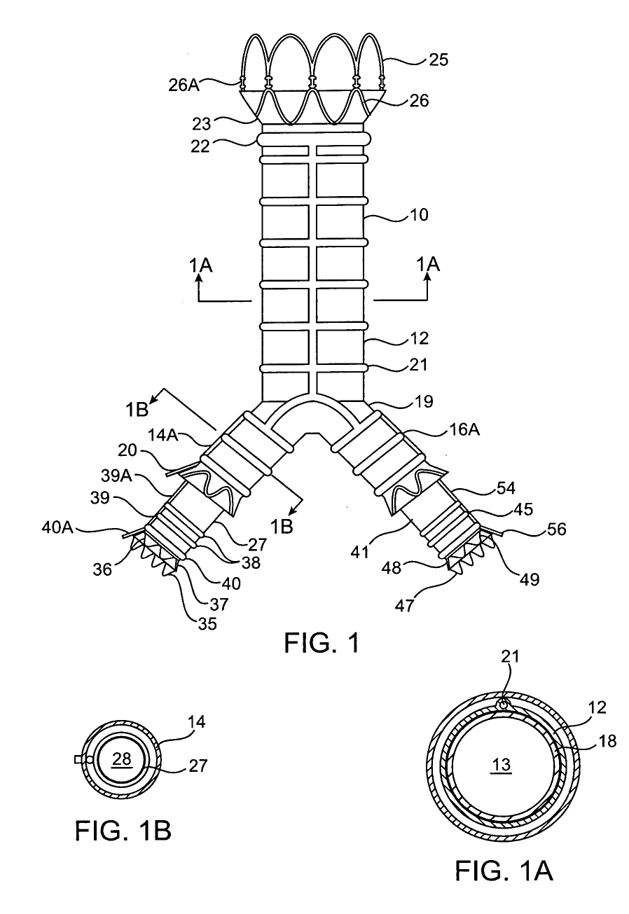

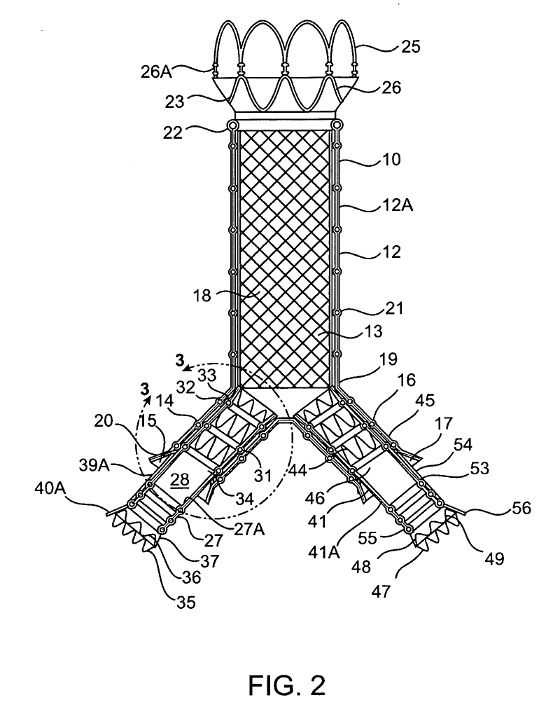

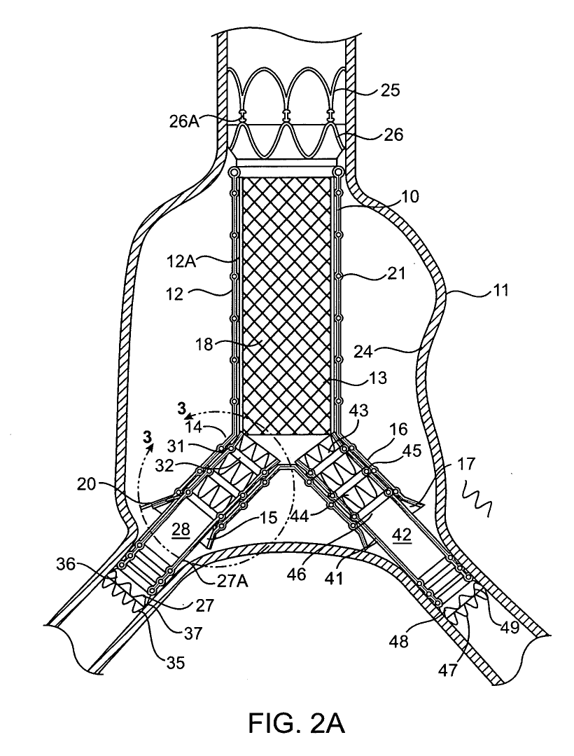

Embodiments of the invention are directed generally to methods and devices for treatment of fluid flow vessels with the body of a patient. Treatment of blood vessels is specifically indicated for some embodiments, and, more specifically, treatment of abdominal aortic aneurysms for others. FIGS. 1-2 illustrate an embodiment of a bifurcated modular endovascular graft or stent-graft 10 for treatment of an abdominal aortic aneurysm 11. The graft 10 is shown deployed within an abdominal aortic aneurysm 11 in FIG. 2A. The graft 10 has a main graft body section 12 with a wall portion 12A that bounds a main fluid flow lumen 13 disposed therein. An ipsilateral attachment element 14 is disposed on a ipsilateral leg 14A that extends distally from a distal portion 19 of the main graft body section 12 and has a ipsilateral port 15 that is in fluid communication with the main fluid flow lumen 13.

A contralateral attachment element 16 is disposed on a contralateral leg 16A that extends distally from the distal portion 19 of the main graft body section and has a contralateral port 17 that is in fluid communication with the main fluid flow lumen 13. The main graft body section 12, ipsilateral leg 14A and contralateral leg 16A form a bifurcated “Y” shaped configuration with the main fluid flow lumen 13 of the main graft body section 12 typically having a larger transverse dimension and area than that of either the ipsilateral port 15 or contralateral port 17. The transverse dimension or diameter of the main fluid flow lumen may be from about 15.0 mm to about 32.0 mm. The transverse dimension or diameter of the ipsilateral and contralateral ports 15 and 17 may be from about 5.0 to about 20.0 mm. The main graft body section 12 may comprise polytetrafluoroethylene (PTFE) or expanded polytetrafluoroethylene (ePTFE). In particular, main graft body section 12 may comprise any number of layers of PTFE and/or ePTFE, including from about 2 to about 15 layers, having an uncompressed layered thickness of about 0.003 inch to about 0.015 inch. Unless otherwise specifically stated, the term “PTFE” as used herein includes both PTFE and ePTFE. Furthermore, the graft body sections of the present invention described herein may comprise all PTFE, all ePTFE, or a combination thereof. Such graft body sections may comprise any alternative biocompatible materials, such as DACRON, suitable for graft applications.

Descriptions of various constructions of graft body sections may be found in commonly-owned U.S. Pat. No. 6,776,604, entitled “Method and Apparatus for Manufacturing an Endovascular Graft Section”, pending U.S. patent application Ser. No. 10/029,584, entitled “Endovascular Graft Joint and Method of Manufacture”, and pending U.S. Patent Application Ser. No. 10/029,559, entitled “Method and Apparatus for Shape Forming Endovascular Graft Material”, all of which were filed on Dec. 20, 2001 to Chobotov et al., the entirety of each of which is incorporated herein by reference.

An optional main expandable stent 18 is disposed within the main graft body section 12 and extends longitudinally within the main graft body section 12 to provide mechanical support to the graft 10. The optional main expandable stent 18 can be mechanically secured to the inside surface of the wall portion of the main graft body section 12, as shown in FIG. 2, or embedded between the layers of PTFE of the main graft body section 12. The elements of the main expandable stent 18 which are configured as a mesh or mesh-like structure may be made from any suitable resilient material such as stainless steel, nickel titanium alloy and the like. The elements of the main expandable stent 18 may have a transverse dimension of about 0.010 inch to about 0.040 inch. The main expandable stent 18 may extend from the distal portion 19 of the main graft body section 12 to the proximal portion 23 of the main graft body section.

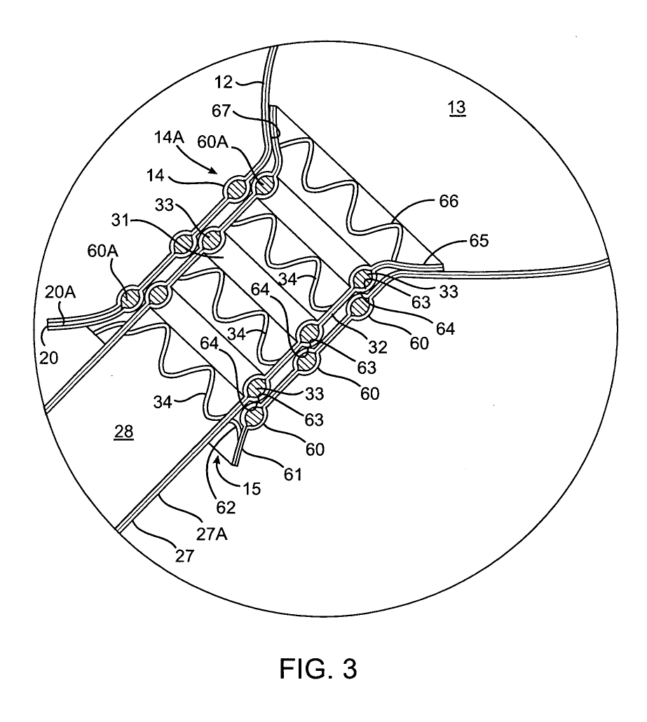

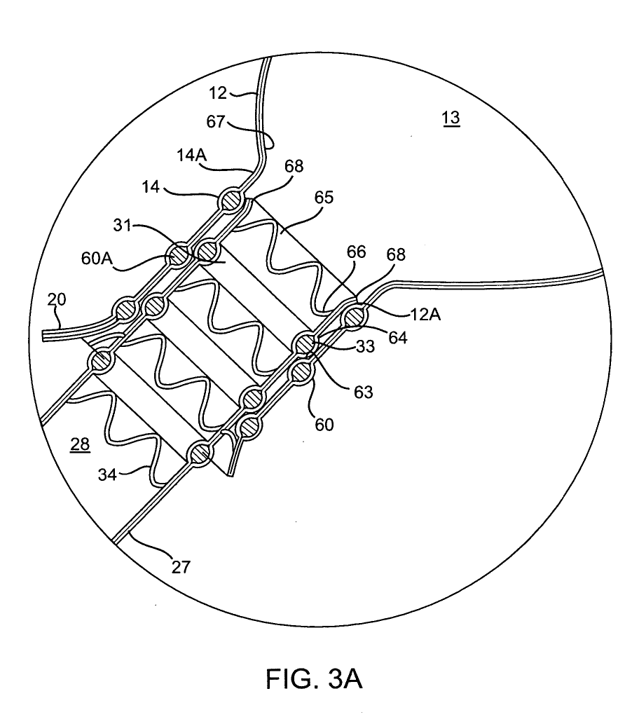

A network of inflatable elements or channels 21 is disposed on the main graft body section 12 which may be inflated under pressure with an inflation material through a main fill port 20 that has a lumen disposed therein in fluid communication with the network of inflatable channels 21. The inflation material may be retained within the network of inflatable channels 21 by a one way-valve 20A (FIG. 3), disposed within the lumen of the main fill port 20. The network of inflatable channels 21 may optionally be filled with a curable fluid in order to provide mechanical support to the main graft body section 12. An inflatable element or cuff 22 is disposed on a proximal portion 23 of the main graft body section 12 and has an outer surface that extends radially from a nominal outer surface of the main graft body section 12. The radial extension of the inflatable cuff 22 from the nominal outer surface of the main graft body section 12 may provide a seal against an inside surface 24 of a blood vessel 11 when the inflatable cuff 22 is in an inflated state. The interior cavity of the inflatable cuff 22 is in fluid communication with the interior cavity of the network of inflatable channels 21 and may have a transverse dimension or inner diameter of about 0.040 inch to about 0.200 inch.

The inflatable cuff 22 and network of inflatable channels 21 may be filled during deployment of the graft 10 with any suitable inflation material that provides outward pressure or a rigid structure from within the inflatable cuff or network of inflatable channels 21. Biocompatible gases or liquids may be used, including curable polymeric materials or gels, such as the polymeric biomaterials described in pending U.S. patent application Ser. No. 09/496,231 filed Feb. 1, 2000, and entitled “Biomaterials Formed by Nucleophilic Addition Reaction to Conjugated Unsaturated Groups” to Hubbell et al. and pending U.S. patent application Ser. No. 09/586,937, filed Jun. 2, 2000, and entitled “Conjugate Addition Reactions for Controlled Delivery of Pharmaceutically Active Compounds” to Hubbell et al. and further discussed in commonly owned pending U.S. patent application Ser. No. 10/327,711, filed Dec. 20, 2002, and entitled “Advanced Endovascular Graft” to Chobotov, et al., each of which is incorporated by reference herein in its entirety.

A proximal expandable stent 25 may be disposed proximally of the main graft body section 12 and is secured to a proximal connector ring 26 which is at least partially disposed in proximal portion 23 of the main graft body section 12. The proximal connector ring 26 has connector elements 26A extending proximally from the proximal connector ring 26 beyond the proximal end of the main graft body section 12 in order to couple or be otherwise secured to mating connector elements of the proximal expandable stent 25. The proximal expandable stent 25 may have a cylindrical or ring-like configuration with the element of the stent being preformed in a serpentine or sine wave pattern within the cylinder as shown in FIGS. 1-2. The elements of the proximal expandable stent 25 may have a thickness of about 0.005 inch to about 0.040 inch. Additional stents may also be disposed at a proximal end of the proximal expandable stent 25 having the same or similar features, dimensions or materials to those of the proximal expandable stent 25. The terms “disposed in” and “disposed on” are used interchangeably throughout the specification. Such terms are meant to include a ring, stent, or other element being coupled to an interior surface of a layer, to an exterior surface of a layer, and between layers.

The proximal expandable stent 25 may be made from a variety of resilient and expandable materials, such as stainless steel, nickel titanium alloy or the like. The proximal expandable stent 25 or additional stents secured to proximal expandable stent 25 may have the same or similar features, dimensions or materials to those of the stents described in commonly owned pending U.S. patent application Ser. No. 10/327,711. The proximal expandable stent 25 may also be secured to the connector ring 26 in the same or similar fashion as described in the incorporated application above.

A ipsilateral graft body section 27 has a ipsilateral fluid flow lumen 28 disposed therein which is bounded by a wall portion 27A of the ipsilateral graft body section 27, as shown in FIG. 3. A first attachment element 31 is disposed on a proximal portion 32 of the ipsilateral graft body section 27 and includes, in the FIG. 3 embodiment, three inflatable elements or circumferential channels 33 and three cylindrical stents 34 disposed in the wall portion 27A of the proximal portion 32 of the ipsilateral graft body section 27. The ipsilateral graft body section 27 may alternatively comprise a lesser or greater number of inflatable elements 33 and stents 34. The cylindrical stents 34 are disposed between the layers of PTFE of the ipsilateral graft body section 27 distally in an axial direction from each of the circumferential inflatable channels 33. The cylindrical stents 34 may also be disposed exterior or interior to the layers of PTFE of ipsilateral graft body section 27. As shown in FIGS. 1 and 2, an ipsilateral distal expandable stent 35 may optionally be secured to a ipsilateral connector ring 36 that is at least partially disposed in the wall portion of the distal portion 37 of the ipsilateral graft body section 27.

As shown in FIGS. 1 and 2, two or more circumferential inflatable channels 38 are disposed on a distal portion 39 of the ipsilateral graft body section proximal of a ipsilateral sealing cuff 40 that is disposed on the distal portion 39 distally of the circumferential inflatable channels 38. More than one ipsilateral sealing cuff 40 may be included on distal portion 39. The ipsilateral sealing element or cuff 40 is disposed proximally of the ipsilateral connector ring 36. The circumferential inflatable channels 38 and ipsilateral sealing cuff 40 are in fluid communication with the circumferential inflatable elements or channels 33 of the first attachment element 31 by an inflatable channel 39A. The circumferential inflatable channels 33 and 38, inflatable channel 39A and ipsilateral sealing cuff 40 can be inflated with an inflation material, such as the inflation materials discussed above, through an ipsilateral fill port 40A. Some or all of the inflatable channels 38 (and similar channels of other components, such as, e.g., ipsilateral graft body section 27 and contralateral graft body section 41 described below) may be disposed circumferentially such as shown in the embodiment of FIG. 1; alternatively, such channels may be disposed in spiral, helical, or other configurations. Examples of channel configurations suitable for embodiments of the present invention are described further in commonly-owned pending U.S. patent application Ser. No. 10/384,103, filed Mar. 6, 2003 and entitled “Kink Resistant Endovascular Graft” to Kari et al., the entirety of which is incorporated herein by reference. It is understood that for all inflatable channels on all components of embodiments of the present invention described herein as circumferential, such channels may alternatively take on any of such aforementioned alternative configurations.

A contralateral graft body section 41 has a contralateral fluid flow lumen 42 disposed therein which is bounded by a wall portion 41A of the ipsilateral graft body section 41, as shown in FIG. 3. A second attachment element 43 is disposed on a proximal portion 44 of the contralateral graft body section 41 and includes three inflatable elements or circumferential channels 45 and three cylindrical stents 46 disposed in the wall portion 41A of the proximal portion 44 of the contralateral graft body section 41. The contralateral graft body section 41 may alternatively comprise a lesser or greater number of inflatable elements 33 and stents 34. The cylindrical stents 46 may be disposed between the layers of PTFE of the contralateral graft body section 41 distally in an axial direction from each of the circumferential inflatable channels 45. The cylindrical stents 46 may also be disposed exterior or interior to the layers of PTFE of contralateral graft body section 41. An optional contralateral distal expandable stent 47 is secured to a contralateral connector ring 48 that is at least partially disposed in the wall portion 41A of the distal portion 49 of the contralateral graft body section 41.

As shown in FIGS. 1 and 2, two or more circumferential inflatable channels 52 are disposed on a distal portion 53 of the contralateral graft body section 41 proximal of a contralateral sealing cuff 55 that is disposed on the distal portion 53 distally of the circumferential inflatable channels 52. More than one contralateral sealing cuff 50 may be included on distal portion 53. The contralateral sealing cuff 55 is disposed proximally of the contralateral connector ring 48. The circumferential inflatable channels 52 and contralateral sealing cuff 55 are in fluid communication with the circumferential inflatable channels 52 of the second attachment element 43 by an inflatable channel 54. The circumferential inflatable channels 45 and 52, inflatable channel 54 and ipsilateral sealing cuff 55 can be inflated with an inflation material, such as the inflation materials discussed above, through a contralateral fill port 56.

Referring to FIG. 3, an enlarged view of a joint between the ipsilateral attachment element 14 and the first attachment element 31 of the ipsilateral graft body section 27 is shown. A flared reinforced portion 61 having an outwardly tapered configuration is disposed on the distal portion of the ipsilateral leg 14A of the main graft body section 12. The flared reinforced portion 61 includes a reinforcing ring 62 which is disposed on the distal portion of the ipsilateral leg 14A. The flared reinforced portion 61 has a generally frustoconical configuration in an outwardly tapered configuration. The flared reinforced portion 61 may provide a guiding function when the ipsilateral graft body section 27 is being advanced into the ipsilateral port 15 during deployment of the graft 10.

Circumferential inflatable channels 60 of the ipsilateral attachment element 14 are shown in an inflated state with an inflation material 60A disposed within the circumferential inflatable channels 60. The configuration of the inflated circumferential inflatable channels 60 of the ipsilateral attachment element 14 includes reduced circumference shoulder portions 63 which intrude into the ipsilateral port 15 and provide a surface for engagement of the mating reduced circumference shoulder portions 64 of the first attachment element 31 as shown.

The mechanical interference or engagement of the reduced circumference shoulder portions 63 and 64 prevent axial movement of the ipsilateral graft body section 27 in a distal direction relative to the ipsilateral attachment element 14. The mechanical interference or engagement of the reduced circumference shoulder portions 63 and 64 would also limit the axial travel of the ipsilateral graft body section 27 in a proximal direction relative to the ipsilateral attachment element 14. Reinforcing stents 34 of the first attachment element 31 of the ipsilateral graft body section 27 provide a resilient surface for seating of the circumferential inflatable channels 60 of the ipsilateral attachment 14 element, help create a seal with the channels 60 and may also prevent intrusion of the circumferential channels 60 into the ipsilateral fluid flow lumen 28.

The inflatable circumferential channels 60 also may provide a seal between the ipsilateral attachment element 14 and an outside surface of the ipsilateral graft body section 27. Likewise, the inflatable circumferential channels 33 of the ipsilateral graft body section 27 may provide a seal between the ipsilateral graft body section 27 and the ipsilateral attachment element by pressing against an inside surface of the ipsilateral port 15 of the ipsilateral attachment element 14.

The proximal portion 32 of the ipsilateral graft body section 27 may include a flared or outwardly tapered reinforced segment 65 disposed proximally of the first attachment element 31. The flared reinforced segment 65 extends to the proximal end of the ipsilateral graft body section 27 and has a flared reinforcing ring 66 that is disposed in the proximal portion 32 of the ipsilateral graft body section 27. The ring 66 will have a generally frustoconical configuration that matches the configuration of the flared reinforced segment 65 and provides a resilient outward radial force of radially compressed or restrained. The flared reinforced segment 65 can mechanically engage a tapered inside surface 67 of the main graft body section 12 to further prevent axial movement of the ipsilateral graft body section 27 in a distal direction relative to the ipsilateral attachment element 14. The flared reinforced segment 65 may also provide a smooth lumen at the transition between the main fluid flow lumen 13 and the ipsilateral fluid flow lumen 28 by providing a smooth tapered lead-in to the ipsilateral fluid flow lumen 28 from the main fluid flow lumen 13.

The joint between the contralateral attachment element 16 and the contralateral graft body section 41 may be carried out in the same or similar fashion to the joint between the ipsilateral attachment element 14 and ipsilateral graft body section 27 described above. In addition, the joint between the contralateral attachment element 16 and the contralateral graft body 41 section may have the same or similar features, such as axial length adjustability, as the joint between the ipsilateral attachment element 14 and ipsilateral graft body section 27 described above.

Referring to FIG. 3A, an enlarged view of the joint between the ipsilateral attachment element 14 and the first attachment element 31 of the ipsilateral graft body section 27 is shown wherein the ipsilateral graft body section 27 has been displaced distally by a length equal to the axial distance between adjacent circumferential inflatable channels 60 of the ipsilateral attachment element 14. As such, the axial length of the axially overlapped portions of the ipsilateral attachment element 14 and first attachment element 31 is less than the length of the axial overlap of the joint illustrated in FIG. 3.

In this configuration, the reduced circumference shoulder portions 63 of the ipsilateral attachment element 14 are again mechanically engaged with the reduced circumference shoulder portions 64 of the first attachment element 31. However, the engagement is shifted such that the distal most circumferential inflatable channel 33 is no longer engaging a circumferential inflatable channel 60 of the ipsilateral attachment element 14. In addition, the flared reinforced segment 65 is disposed within the ipsilateral attachment element 14 and is pressing radially outward against an inside surface of the wall portion 12A of the ipsilateral leg 14A and is also partially mechanically engaging a reduced circumference shoulder portion 68 of one of the circumferential inflatable channels 60 as shown in FIG. 3A.

Deployment of the bifurcated modular endovascular graft 10 may be carried out by any suitable method, including techniques and accompanying apparatus as disclosed in commonly owned U.S. Pat. No. 6,761,733 to Chobotov et al., pending U.S. patent application Ser. No. 10/686,863 entitled “Delivery Systems and Methods for Bifurcated Endovascular Graft” to Chobotov et al., filed Oct. 16, 2003 the entirety of both are incorporated herein by reference. In one deployment method, the main graft body section 12 is advanced in the patient's vessel 11, typically in a proximal direction from the ipsilateral iliac artery, to a desired site of deployment, such as the abdominal aorta 11 shown in FIG. 2A, in a constrained state via a catheter or like device having a low profile for ease of delivery through the patient's vasculature. At the desired site of deployment, the main graft body section is released from a constrained state and the stent 25 (and optional stent 18, if present) is allowed to expand and secure a portion of the main graft body section 12 to the patient's vasculature. Thereafter, the network of inflatable channels 21 may be partially or fully inflated by injection of a suitable inflation material into the main fill port 20 to provide rigidity to the network of inflatable channels 21 and the main graft body section 12, in addition to providing a seal between the inflatable cuff 22 and the inside surface of the abdominal aorta 11. This inflation step also fills the circumferential inflatable channels 60 of the ipsilateral attachment element 14 and creates a main graft body section configuration having reduced circumference shoulder portions 63. Although it is desirable to partially or fully inflate the network of inflatable channels 21 of the main graft body section 12 at this stage of the deployment process, such inflation step optionally may be accomplished at a later stage if necessary.

The ipsilateral graft body section 27 is then advanced into the patient's vasculature, again typically in a proximal direction from the ipsilateral iliac in a constrained state via a catheter or like device until the first attachment element 31 is disposed within the ipsilateral attachment element 14 of the main graft body section 12. The ipsilateral graft body section 27 is then released from the constrained state and the circumferential inflatable channels 33 of the first attachment element 31, the inflatable channels 38 and the ipsilateral sealing cuff 40 may then all be inflated by injection of inflation material into the ipsilateral fill port 40A. This causes the inflatable channels 33 of the first attachment element 31 to engage the circumferential inflatable channels 60 of the ipsilateral attachment element 14. The engagement of the ipsilateral attachment element 14 and first attachment element 31 is such that a seal is created between the elements 14 and 31. In addition, the engagement substantially prevents axial displacement of movement to separate the ipsilateral graft body section 27 in a distal direction relative to the ipsilateral attachment element 14 of the main graft body section 12. Both the main fill port 20 and ipsilateral fill port may include a valve, such as a one way valve 20A, that allows the injection of inflation material but prevents the escape thereof. The same or similar procedure is carried out with respect to the deployment of the contralateral graft body section in the contralateral attachment element 16 of the main graft body portion 12. Note that in the embodiment shown in FIG. 1, the circumferential inflatable channels 52 of the contralateral attachment element 16 are in fluid communication with the main fill port and will be inflated into an inflated state at the same time the rest of the main graft body section 12 is inflated, although other configurations in which a separate fill port for the contralateral graft body section are contemplated.

As discussed above, the inflation channels 21 of main graft body section 12, channels 38 of ipsilateral graft body section 27 and channels 52 of contralateral graft body section 41 may be inflated in any sequence and in any number of partial steps until the desired level of inflation is achieved, to effect the desired clinical result. As such, the deployment and inflation sequence described above is but one of a large number of sequences and methods by which the embodiments of the present invention may be effectively deployed.

The various embodiments of the present invention may also be used for deploying and joining multiple sections of non-bifurcated endoprostheses, which are useful, for example, in treating TAAs. Examples of such non-bifurcated devices, their delivery systems and methods for delivery are described in commonly-owned U.S. Pat. Nos. 6,331,191, 6,395,019, 6,733,521 to Chobotov et al. and pending U.S. patent application Ser. No. 10/327,711, the entirety of each of which are incorporated herein by reference. Two or more sections of tubular endoprostheses may be joined using the technologies described herein to achieve the desired length for effectively treating TAAs, aortic dissections, and other conditions in the thoracic or other sections of the aorta or other vessel in which a non-bifurcated endoprosthesis is indicated.

Referring to FIG. 4, an alternative embodiment of a joint between an ipsilateral attachment element 71 and first attachment element 72 of an ipsilateral graft body section 73 having a fluid flow lumen 73A disposed therein is shown. In this embodiment, the ipsilateral attachment element includes a plurality of resilient members in the form of cylindrical stents 74 disposed in the wall portion 75 of the substantially tubular ipsilateral attachment element 71. The cylindrical stents 74 provide for enhanced engagement of the circumferential inflatable channels 76 which press in an outward radial direction into the wall portion 75 when the inflatable channels 76 are in an inflated state.

Inflated circumferential inflatable channels 76 have reduced circumference shoulder portions 77 that engage reduced circumference shoulder portions 78 of the ipsilateral attachment element 71. Shoulder portions 78 are created by the outward pressure and displacement of the wall portion 75, which form recessed pockets in the wall portion 75 due to outward pressure from the circumferential inflatable channels 76. The strength and resilience of the reduced circumference shoulder portions 78 of the ipsilateral attachment element 71 is enhanced by the cylindrical stents 74 which provide greater resistance to outward displacement of the wall portion 75 than adjacent areas of the wall portion that do not include reinforcing stents 74. A flared reinforced segment 79 is disposed at the distal end of the first attachment element 72 and engages a tapered portion 80 of the ipsilateral attachment element 71 of the main graft body section 12. The flared reinforced segment 79 may include a resilient ring 81 disposed in the wall portion 75 of the flared reinforced segment 79 that is resistant to radial compression and expansion.

The engagement of the ipsilateral attachment element 71 and first attachment element 72 is such that a seal is created between the elements 71 and 72. In addition, the engage ment substantially prevents axial displacement of movement or separation of the ipsilateral graft body section 73 in a distal direction relative to the ipsilateral attachment element 71 of the main graft body section 12 and provides for a length adjustability in a fashion similar to the embodiment described in conjunction with FIG. 3A.

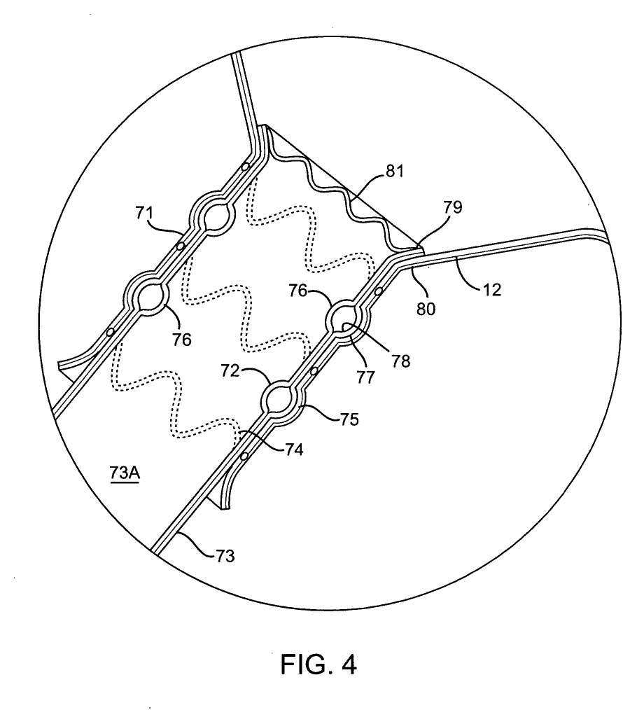

Referring to FIG. 5, an alternative embodiment of a joint between an ipsilateral attachment element 83 and first attachment element 84 of an ipsilateral graft body section 85 having a fluid flow lumen 85A disposed therein is shown. In this embodiment, the ipsilateral attachment element 83 includes a plurality of recessed circumferential pockets 86 pre-formed in a wall portion 86A of the substantially tubular ipsilateral attachment element 83. The recessed circumferential pockets 86 provide for enhanced engagement of the circumferential inflatable channels 87 that press in an outward radial direction into the recessed circumferential pockets 86 when the inflatable channels 87 are in an inflated state.

When inflated circumferential inflatable channels 87 have reduced circumference shoulder portions 88 that engage reduced circumference shoulder portions 89 of the recessed circumferential pockets 86 of the ipsilateral attachment element 83. A flared reinforced segment 90 is disposed at the distal end of the first attachment element 84 and engages a tapered portion 91 of the ipsilateral attachment element 83 of the main graft body section 12. The flared reinforced segment 90 may include a resilient ring 92 disposed in the wall portion 86A of the flared reinforced segment 90 that is resistant to radial compression and expansion which provides further enhancement of the joint between the ipsilateral attachment element 83 and first attachment element 84.

The engagement of the ipsilateral attachment element 83 and first attachment element 84 is such that a seal is created between the elements 83 and 84. In addition, the engagement substantially prevents axial displacement of movement or separation of the ipsilateral graft body section 85 in a distal direction relative to the ipsilateral attachment element 83 of the main graft body section 12.

Referring to FIG. 6, an enlarged view of the FIG. 5 embodiment of a joint between the ipsilateral attachment element 83 and the first attachment element 84 of the ipsilateral graft body section 85 is shown wherein the ipsilateral graft body section 85 has been displaced distally by a length equal to the axial distance between adjacent circumferential inflatable channels 87 of the first attachment element 84. As such, the axial length of the axially overlapped portions of the ipsilateral attachment element 83 and first attachment element 84 is less than the length of the axial overlap of the joint illustrated in FIG. 5.

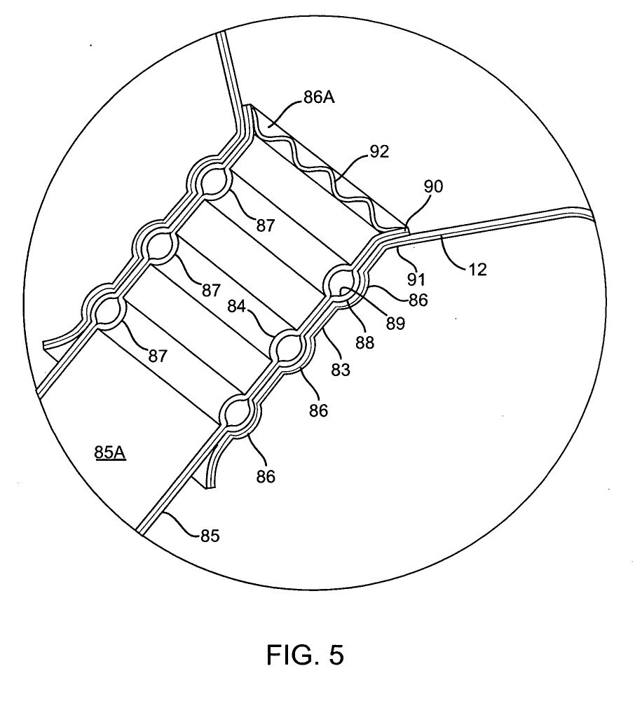

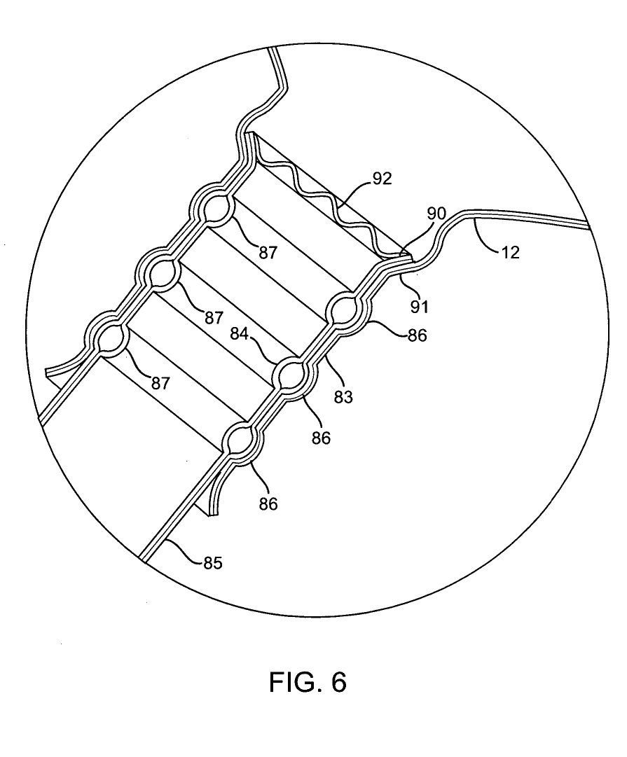

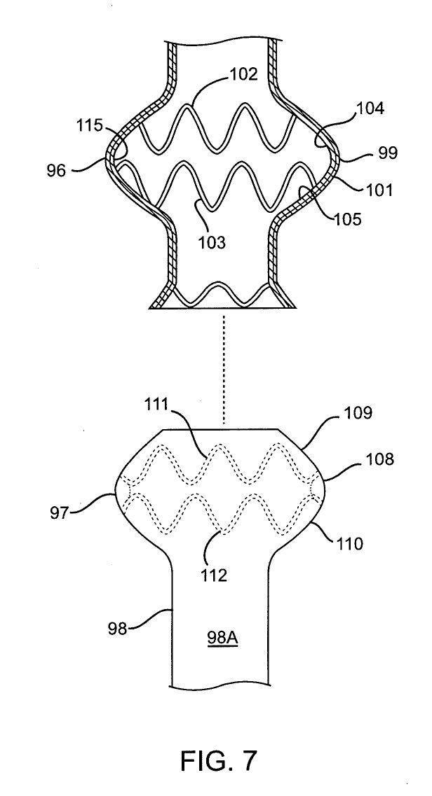

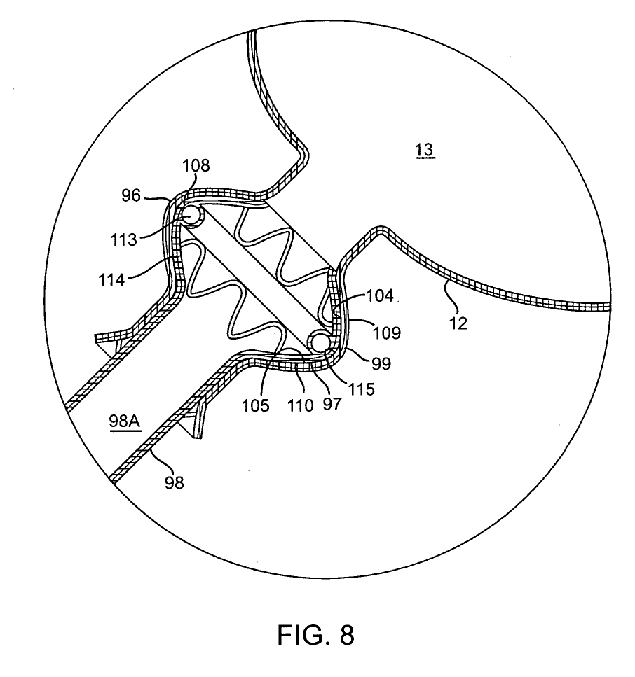

Referring to FIGS. 7 and 8, an alternative embodiment of an ipsilateral attachment element 96 is shown axially aligned with an alternative embodiment of a first attachment element 97 of an ipsilateral graft body section 98 having an ipsilateral fluid flow lumen 98A. In this embodiment, a large reinforced recessed pocket 99 is formed in the wall portion 101 of the ipsilateral attachment element 96. The reinforced recessed pocket 99 has a proximal reinforcing stent 102 and a distal reinforcing stent 103 disposed in the ipsilateral attachment element 96. The proximal reinforcing stent 102 and the distal reinforcing stent 103 may be attached to each other or they may be spaced from each other. The reinforcing stents 102 and 103 provide a resistance to radial compression and expansion that stabilizes the nominal configuration of the reinforced recessed pocket 99. The reinforced recessed pocket 99 also has a proximal reduced circumference shoulder portion 104 and a distal reduced circumference shoulder portion 105 for engagement by the first attachment element 97 of the ipsilateral graft body section 98.

The first attachment element 97 has an enlarged segment 108 with a proximal reduced circumference shoulder portion 109 and a distal reduced circumference shoulder portion 110. The proximal reduced circumference shoulder portion 109 is reinforced by a proximal reinforcing stent 111 that is disposed in the first attachment element 97. The distal reduced circumference shoulder portion is reinforced by a distal reinforcing stent 112 that is also disposed in the first attachment element 97 distal of the stent 111. The reinforcing stents 111 and 112 provide a configuration that resists compressive forces that alter the nominal shape or configuration of the first attachment element 97. The first attachment element 97 also includes a circumferential inflatable channel 113 disposed in the wall portion 114 of the enlarged segment 108 that may be inflated with a pressurized inflation material, such as the inflation materials discussed above, in order to provide further resistance to compressive forces and provide an outward radial force against an inside surface 115 of the ipsilateral attachment element 96.

FIG. 8 illustrates the first attachment element 97 disposed within and captured by the reinforced recessed pocket 99 of the ipsilateral attachment element 96. In this configuration, the proximal reduced circumference shoulder portion 104 and distal reduced circumference shoulder portion 105 of the reinforced recessed pocket 99 engage the proximal reduced circumference shoulder portion 109 and distal reduced circumference shoulder portion 110 of the first attachment element 97, respectively. In the engaged state, the enlarged segment of the ipsilateral graft body section is captured by the reinforced recessed pocket 99 of the ipsilateral attachment element 96 and axial movement of the ipsilateral graft body section 98 relative to the ipsilateral attachment element 96 and main graft body section 12 is prevented. In addition, the outward radial pressure of the circumferential inflatable channel 113 in an inflated state against the inside surface 115 of the reinforced recessed pocket 99 creates a seal between the fluid flow lumen 98A of the ipsilateral graft body section 98 and the main fluid flow lumen 13 of the main graft body section 12.

The first attachment element may be deployed in the reinforced recessed pocket 99 of the ipsilateral attachment element 96 by positioning the enlarged segment 108 of the first attachment element 97 within the reinforced recessed pocket 99 with the enlarged segment 108 in a radially constrained state. Thereafter, the radial constraint on the enlarged segment 108 is removed and the enlarged segment allowed to expand into the reinforced recessed pocket 99.

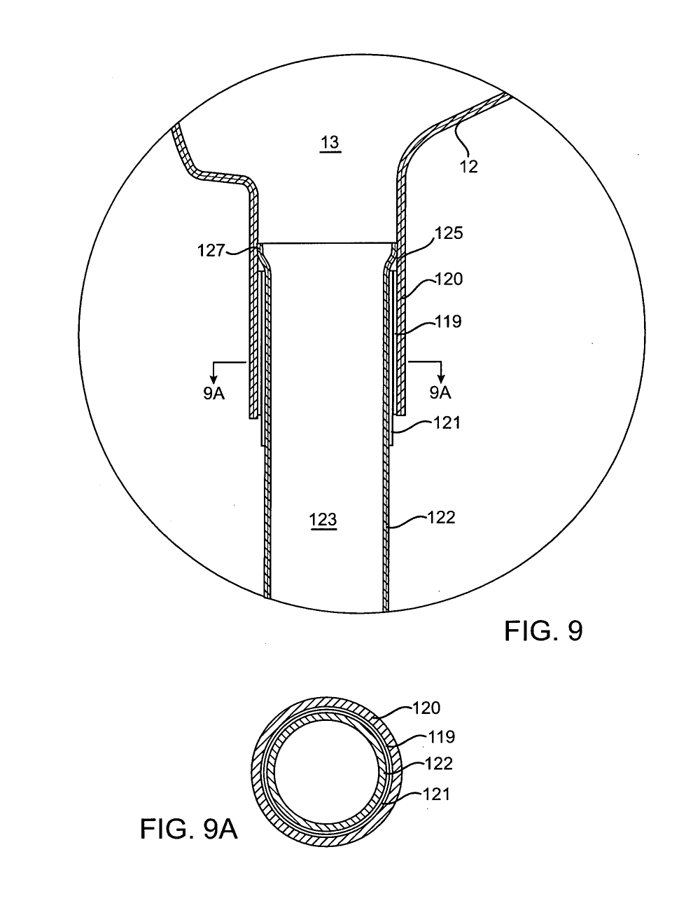

FIGS. 9 and 9A illustrate another alternative embodiment of an ipsilateral attachment element 119 disposed on an ipsilateral leg 120 of a main graft body section 12 that is secured to a first attachment element 121 of an ipsilateral graft body section 122. The ipsilateral graft body section 122 has an ipsilateral fluid flow lumen 123 disposed therein. In this embodiment, the ipsilateral attachment element 119 includes a surface having a plurality of flexible hooks 124 adjacent each other, as shown in FIG. 11, over an area that may be completely disposed about an inner surface 125 of the ipsilateral leg 120.

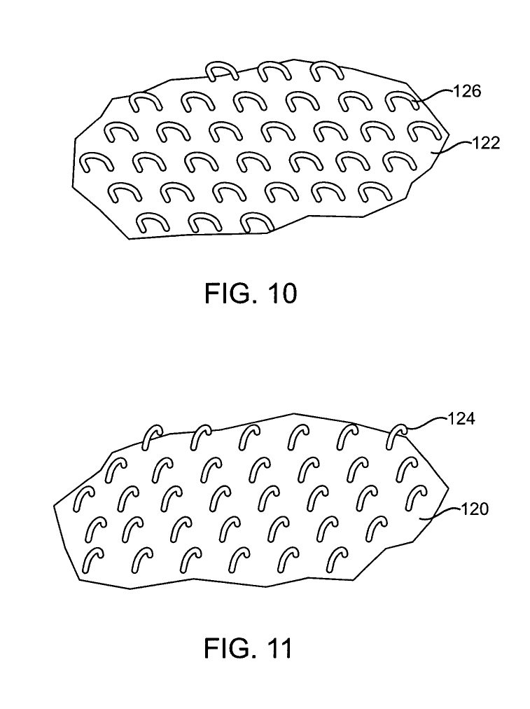

The first attachment element 121 includes a plurality of flexible loops 126 disposed adjacent each other, as shown in FIG. 10, over an area that may be completely disposed about an outer surface of the ipsilateral graft body section 122 in the area covered by the first attachment element 121. The flexible hooks 124 mechanically engage and retain the flexible loops 126 when the surfaces of the ipsilateral attachment element 119 and first attachment element 121 are pressed together, as shown in FIGS. 9 and 9A. This configuration mechanically secures the ipsilateral graft body section 122 to the main graft body section 12 and substantially prevents axial movement of the ipsilateral graft body section 122 relative to the main graft body section 12.

It should be noted that the relative position of the plurality of flexible hooks 124 and flexible loops 126 could be reversed with the same advantage achieved. So long as the surfaces of the ipsilateral attachment element 119 and first attachment element 121 are mutually cohesive, specifically, mutually mechanically cohesive so as to prevent shear displacement, the same or similar result may be achieved. For some embodiments, the length of the flexible hooks may be from about 0.020 inch to about 0.050 inch. The length of the flexible loops may be from about 0.020 inch to about 0.050 inch.

The flared proximal end 127 of the first attachment element 121, which may also be reinforced with an appropriately sized stent (not shown), may provide a smooth fluid flow transition from the main fluid flow lumen 13 to the ipsilateral fluid flow lumen 123. In addition, the flared proximal end 127 may exert an outward radial force against the inside surface of the ipsilateral leg 120 and provide a seal between the main fluid flow lumen 13 and the ipsilateral fluid flow lumen 123.

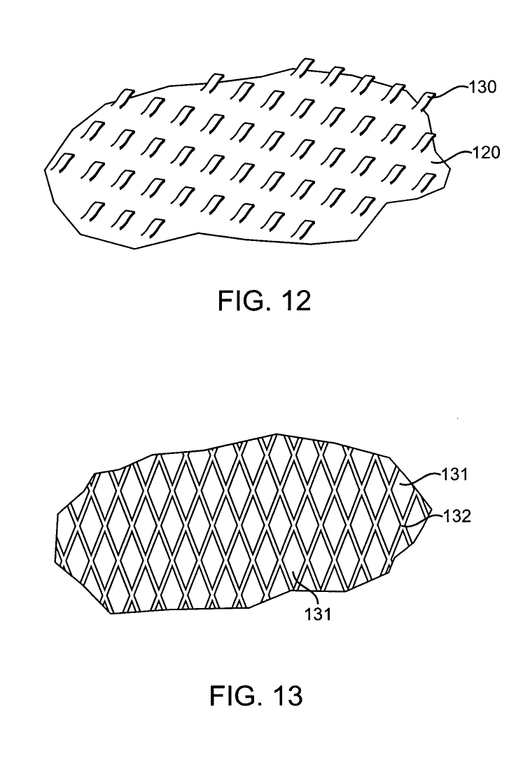

FIGS. 12 and 13 illustrate an alternative embodiment of surfaces that could be used together for either the ipsilateral attachment element 119 or the first attachment element 121. FIG. 12 illustrates a surface having a plurality of pins 130 extending substantially perpendicularly from the surface 120 and configured to mechanically engage the apertures 131 of the mesh 132 and prevent shear displacement when the surfaces are pressed together. As the surfaces of FIGS. 12 and 13 are not mutually cohesive, it may be necessary to provide a biasing member, such as an expandable stent or inflatable cuff (not shown) in the wall of the first attachment element 121 to provide an outward radial force pressing the surfaces together.

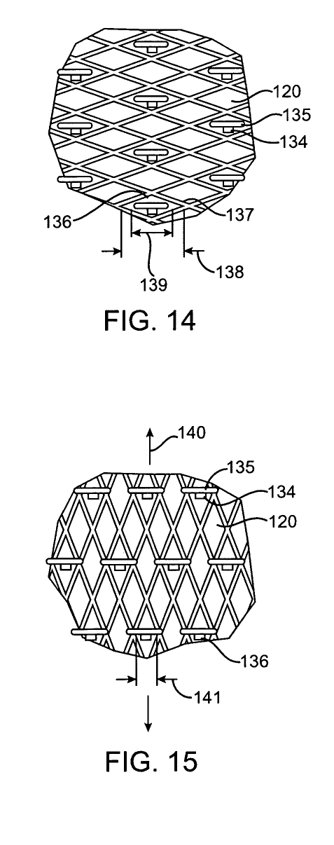

FIGS. 14 and 15 illustrate an embodiment of surfaces that may be activated to be mutually cohesive, and prevent relative shear displacement therebetween. FIG. 14 shows a surface of the ipsilateral attachment element 120 having a plurality of buttons 134 having an enlarged head portion 135 disposed on an outer end of the buttons 134. The enlarged head portion 135 of the buttons 134 are passed through apertures 136 of a convertible mesh 137 that makes up the first attachment element 121. When the convertible mesh 137 is in a circumferentially restrained or low profile state, the axial dimension 138 of the apertures 136 will readily pass an axial dimension 139 of the enlarged head portion 135 of the buttons 134. However, when the convertible mesh is expanded in a circumferential orientation as indicated by arrows 140 in FIG. 15, the axial dimension 141 of the apertures 136 is reduced such that the enlarged head portion 135 is captured and mechanically secured to the convertible mesh 137.

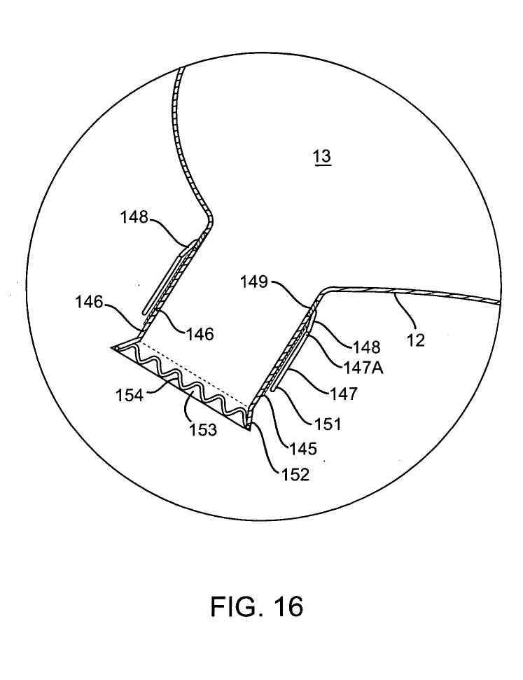

Referring to FIGS. 16-19, an alternative embodiment of a joint between a main graft body section 12 and an ipsilateral graft body section 144 of a modular endovascular graft is illustrated. FIG. 16 shows an ipsilateral attachment element 145 disposed in an outside surface of an ipsilateral leg 146 of the main graft body section 12. A radial compression member in the form of a cylindrical stent 147 is disposed about at least a portion of the ipsilateral attachment element 145 and is secured to the ipsilateral leg 146 at a proximal end 147A of the cylindrical stent 147 by connector elements 148 which are secured to a connector ring 149 which is at least partially disposed in the wall portion of the ipsilateral leg 146. The distal end or free end 151 of the cylindrical stent 147 is not secured to the ipsilateral leg 146 and may freely expand and contract in a radial orientation. A reinforced flared segment 152 is disposed at the distal end 153 of the ipsilateral leg 146 and includes an outwardly tapered segment tapering to an increased transverse dimension distally. A reinforcing ring 154 is disposed in the reinforced flared segment 152.

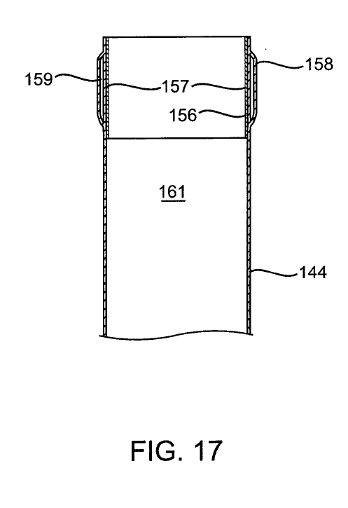

FIG. 17 illustrates the ipsilateral graft body section 144 partially broken away. The proximal portion 156 of the ipsilateral graft body section 144 includes a first attachment element 157 disposed on an inside surface of the wall portion of the ipsilateral graft body section 144. An inflatable cuff 158 is disposed about the proximal portion 156 at least partially over the axial section of the ipsilateral graft body section 144 that includes the first attachment element 157. The inflatable cuff 158 has a cavity 159 disposed therein that may be inflated by a fill port (not shown) through an inflatable channel (not shown) with any suitable inflation material, such as the inflation materials discussed above.

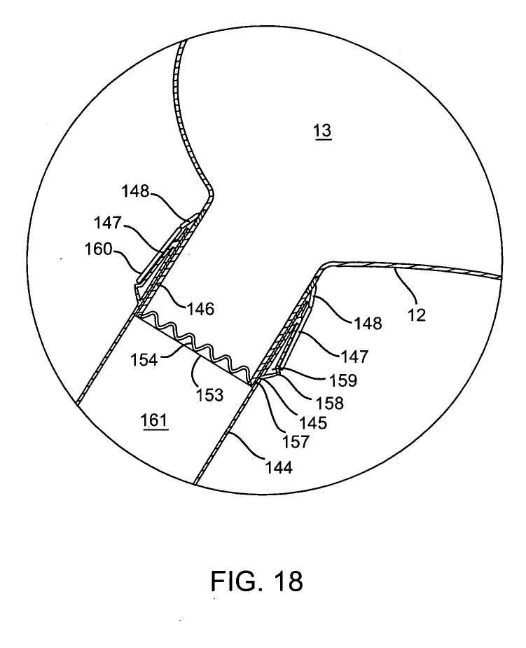

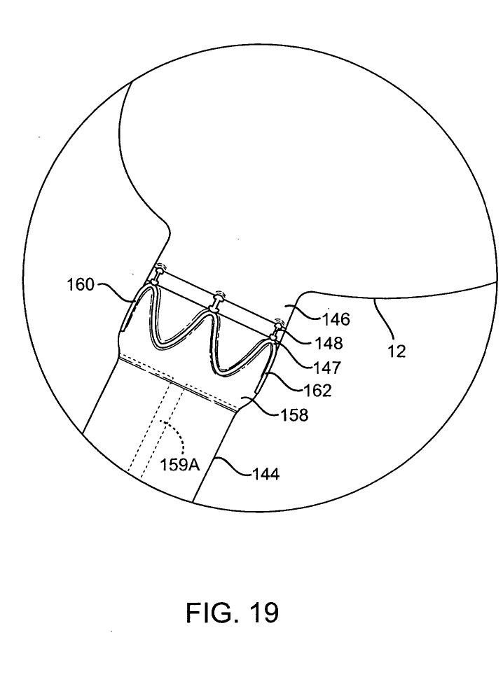

FIGS. 18 and 19 illustrate a sectional view of a joint 160 between the main graft body section 12 and the ipsilateral graft body section 144 wherein the main fluid flow lumen 13 is in fluid communication with and sealed to a fluid flow lumen 161 of the ipsilateral graft body section 144. The joint 160 includes at least portions of the ipsilateral attachment element 145 secured to the first attachment element 157 by compression of the surfaces of the ipsilateral attachment element 145 and first attachment element 157 together.

The ipsilateral attachment element 145 and first attachment element 157 may be mutually mechanically cohesive or otherwise configured to resist shear displacement when pressed together. Suitable combinations of surfaces, such as those discussed above with regard to FIGS. 9-15, may be used for the ipsilateral attachment element 145 and first attachment element 157. For example, an array of flexible hooks 124, as shown in FIG. 11, could be used for the ipsilateral attachment element in conjunction with an array of flexible loops 126, as shown in FIG. 10, for the first attachment element 157.

The mating of the ipsilateral attachment element 145 and first attachment element 157 is enhanced by the inward radial compression on the joint 160 produced by inflation of the inflatable cuff 158. The inflatable cuff 158 expands upon inflation as the cavity 159 fills with inflation material, however, expansion in an outward radial orientation is constrained by the stent 147 which is at least partially disposed over the cuff 158. As such, inflation of the inflatable cuff 158 applies radial compression on the joint 160 which enhances the strength of the joint 160. It should be noted that the same or similar effect could be achieved without the inflatable cuff 158 if the stent 147 was appropriately sized and configured to apply inward radial compression on the joint 160 when in a relaxed or compressed state. The joint 160 as shown in FIG. 19 also includes added strength from the molding of the inflatable cuff 158 about the element 162 of the stent 147. The molding of the cuff 158 about the stent 147 provides an additional mechanical interlock between the proximal portion 156 of the ipsilateral graft body section 144 and the ipsilateral leg 146 of the main graft body section 12.

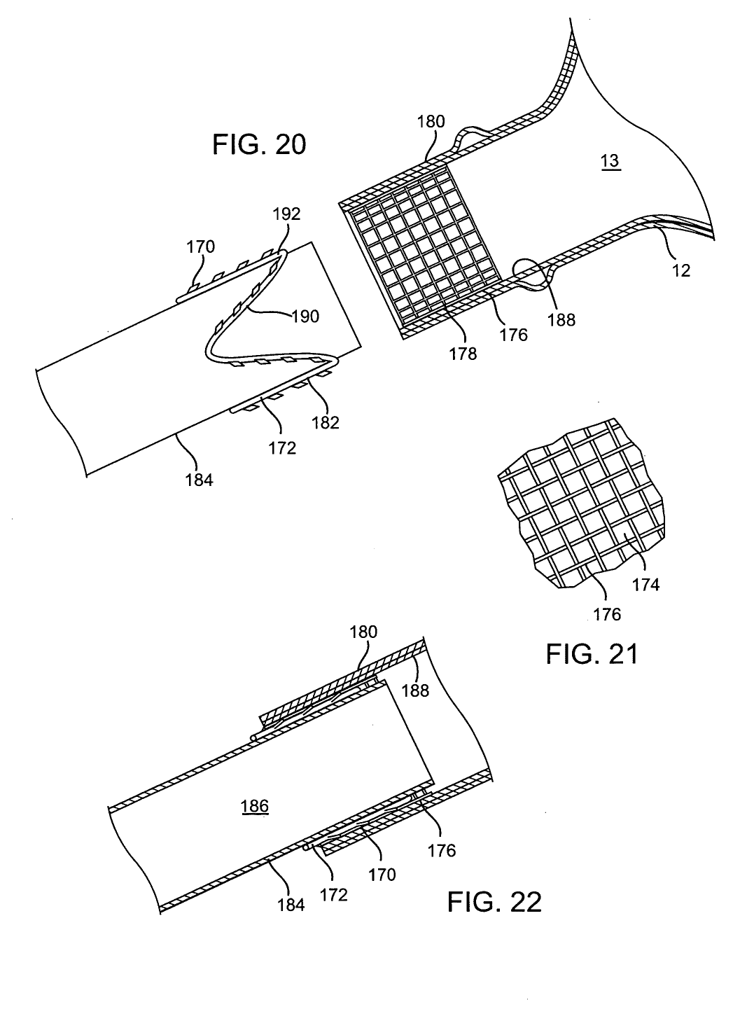

FIGS. 20-22 show alternative embodiments of attachment elements of graft body sections wherein protuberances 170 of an expandable cylindrical member 172 are configured to engage the openings 174 of a mesh 176 or similar structure. An ipsilateral attachment element 178 disposed on an ipsilateral leg 180 of a main graft body section 12 is securable to a first attachment element 182 of an ipsilateral graft section 184 as shown in FIG. 22. The ipsilateral graft section 184 has an ipsilateral fluid flow lumen 186 disposed therein. In this embodiment, the ipsilateral attachment element 178 includes a surface having a mesh structure 176 with a plurality of openings or apertures 174. An enlarged view of a portion of an embodiment of the mesh structure 176 is shown in FIG. 21. The mesh structure 176 may be disposed over and secured to a substantial area of the ipsilateral leg 180 and may be completely disposed about an inner surface 188 of the ipsilateral leg 180. The mesh structure 176 may be secured to the inner surface 188 by any suitable means, such as adhesive bonding, mechanical capture by graft wall portions, or the like.

The first attachment element 182 includes the expandable cylindrical member 172 which has a plurality of protuberances 170 disposed adjacent each other, as shown in FIG. 20. The protuberances 170 extend in an outward radial direction from the expandable cylindrical member 172 and are spaced over a substantial area of the expandable cylindrical member 172. The protuberances 170 are sized and spaced so as to engage the openings 174 of the mesh structure 176 of the ipsilateral attachment element 178 when the surfaces of the ipsilateral attachment element 178 and first attachment element 182 are pressed together, as shown in FIG. 22. In one embodiments the surfaces of the attachment elements 178 and 182 are pressed together by an outward radial force exerted by the expandable cylindrical member 172, which may be balloon expandable, self-expanding or the like. The outward radial force of the expandable cylindrical member 172 may also serve to seal the inner lumen 186 of the ipsilateral graft section 184 to the inner lumen 13 of the main graft section 12. The protuberances 170 may be completely disposed about an outer surface of the expandable cylindrical member 172 and may be cut into the material of the expandable cylindrical member 172 or added to the structure of the expandable cylindrical member by bonding, welding or any other suitable means.

The expandable cylindrical member 172 may be made from a thin element 190 which is formed into the undulating cylindrical pattern as shown in the embodiment of FIGS. 20-22. The structure of the expandable cylindrical member 172 may be made from a cut tube or formed from a thin element or wire of expandable material such as stainless steel, nickel titanium alloy or the like. The expandable cylindrical member may be secured to the ipsilateral graft section 184 by any suitable means such as adhesive bonding, mechanical capture by portions of the graft section wall, or the like. This joint between the ipsilateral attachment element 178 and first attachment element 182 mechanically secures the ipsilateral graft section 184 to the main graft body section 12 and prevents axial movement of the ipsilateral graft section 184 relative to the main graft body section 12. For some embodiments, the length of the protuberances 170 in an outward radial direction from a nominal outer surface 192 of the expandable cylindrical member 172 may be from about 0.005 to about 0.050 inch. A transverse dimension of the openings 174 of the mesh structure 176 may be from about 0.020 to about 0.050 inch for some embodiments.

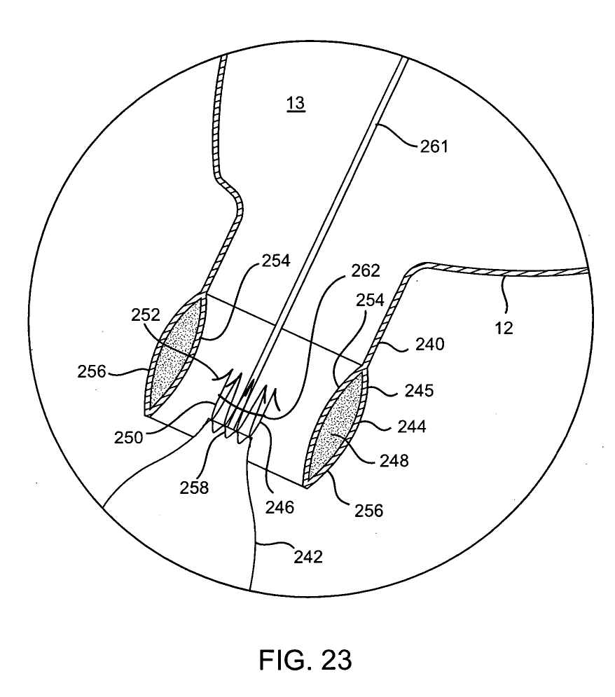

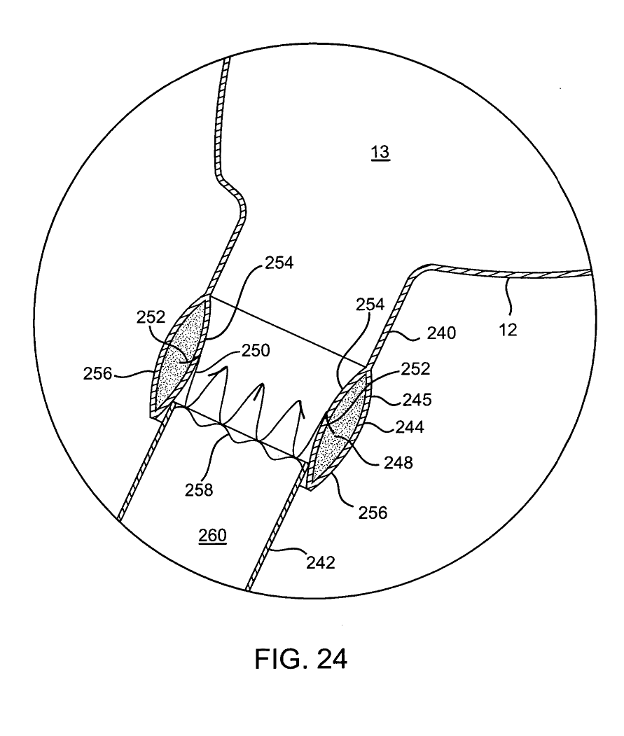

FIGS. 23 and 24 illustrate another alternative embodiment of a junction between an ipsilateral leg 240 of a main graft body section 12 and an ipsilateral graft body section 242. The junction, as shown in FIG. 24, is formed by an ipsilateral attachment element 244 disposed on the ipsilateral leg 240 of a main graft body section 12 and a first attachment element 246 disposed on the ipsilateral graft body section 242. The ipsilateral attachment element 244 includes a circumferential inflatable cuff 245 that is filled with an inflation material 248. The first attachment element 246 includes an expandable member or stent device 250 disposed on the ipsilateral graft body section 242 which is configured to expand and engage an inside surface of the inflatable cuff 245 of the ipsilateral attachment element 244.

The expandable member 250 may also include barbs 252 which are configured to extend radially from the expandable member 250 and protrude through an inner wall 254 of the inflatable cuff 245 and into the inflation material 248. In some embodiments, the length and configuration of the barbs 252 are chosen so as to penetrate the inner wall 254 and into the inflation material 248 without penetrating an outer wall 256 of the inflatable cuff 245. The inflation material 248 shown in FIGS. 23 and 24 may be curable such that it serves as a substantially rigid anchoring platform for the expandable member 250 to be secured to in addition to providing a sealing function whereby the outer wall 256 may be sealed against an inside surface of a patient's vessel. This configuration mechanically secures the ipsilateral graft body section 242 to the main graft body section 12 and substantially prevents axial movement of the ipsilateral graft body section 242 relative to the main graft body section 12. The barbs 252 may be configured to extend in a radial orientation that is substantially orthogonal to a longitudinal axis of the ipsilateral graft body section 242, or the barbs 252 may be configured to extend at an angled bias either in the proximal or distal direction, as shown in FIG. 24.

In addition to an expandable member 250, the first attachment element 246 of the ipsilateral graft body section 242 may also include a connector ring 258 disposed in the PTFE material of the ipsilateral graft body section 242. The connector ring 258 may provide an anchor and strain relief function for the expandable member 250 which is secured thereto. The connector ring 258 may be secured inside, outside or within the wall of the ipsilateral graft body section 242. The portion of the ipsilateral graft body section 242 that surrounds the connector ring 258 may be flared or tapered to provide a smooth fluid flow transition from the main fluid flow lumen 13 to the ipsilateral fluid flow lumen 260 of the ipsilateral graft body section 242.

As shown in FIG. 23, during deployment, the main graft body section 12 may be inserted into the patient's vasculature with the inflatable cuff 245 in an uninflated state for low profile delivery. Once the main graft body section has been positioned within the patient's vasculature, the inflatable cuff 245 may then be inflated with inflation material 248 which may then be cured to form a substantially rigid body with sufficient tensile properties to anchor barbs 252 of the expandable member 250. Once the inflatable cuff 245 has been deployed and filled, the ipsilateral graft body section 242 may then be inserted into the ipsilateral attachment element 244 over a guidewire or similar device 261 with the expandable member 250 in a contracted state. The expandable member 250 is restrained in a contracted state by a restraining element 262 disposed about the expandable member 250. Once the expandable member 250 is prQoperly positioned with respect to the inflatable cuff 245, the restraining element 262 may then be removed so as to allow the expandable member 250 to expand and engage the inside surface 254 of the inflatable cuff 244. As the expandable member 250 expands, the barbs 252 radially extend and penetrate the inner wall 254 of the inflatable cuff 244 and the cured material 248 disposed within the inflatable cuff 244 so as to form the junction between the ipsilateral leg 240 and ipsilateral graft body section 242. Note that expandable member 250 may be self-expandable as described above or may be expandable by the application of a suitable force, such as with a balloon-expandable material. In the latter case, restraining element 262 may therefore be an optional feature. As such, any suitable metallic or polymeric material, such as stainless steel, nitinol and the like, may be used for expandable member 250.

While particular forms of embodiments of the invention have been illustrated and described, it will become apparent that various modifications may be made without departing from the spirit and scope of the invention. For example, while the illustrated endovascular grafts have a main graft body section and an ipsilateral graft body section and a contralateral graft body section, other embodiments of the present invention may only include one of the ipsilateral graft body section and the contralateral graft body sections. In such embodiments, the ipsilateral graft body section or the contralateral graft body section may be integrally formed with the main graft body section, and the other of the ipsilateral graft body section or contralateral graft body section may be attachable to the main graft body section. In addition, all of the embodiments of the present invention described herein may be used in non-bifurcated endoprosthesis applications to join or attach two or more such graft sections, especially for treating conditions in the thoracic aorta.

Moreover, while the illustrated embodiments have the ipsilateral graft body section and contralateral graft body section at least partially positioned within the ipsilateral leg and contralateral leg of the main graft body portion, it should be appreciated that in alternative embodiments it may be possible to have the ipsilateral leg and contralateral leg of the main graft body portion at least partially positioned within the ipsilateral graft body section and contralateral graft body section.

Accordingly, it is not intended that the invention be limited by the foregoing exemplary embodiments.

1. A modular endovascular graft, comprising:
a first graft body section having a first fluid flow lumen bounded by a first wall portion, a first attachment element disposed on the first wall portion and an inflatable cuff surrounding the first fluid flow lumen and extending radially from the first wall portion when in an inflated state; and
a second graft body section having a second fluid flow lumen bounded by a second wall portion, a second attachment element disposed on the second wall portion which is configured to be secured to the first attachment element with the first fluid flow lumen sealed to the second fluid flow lumen.

2. The modular endovascular graft of claim 1 wherein the first graft body section further comprises a network of inflatable channels distributed over the first graft body section and in fluid communication with the inflatable cuff to provide structural rigidity and support to the first graft body section when the network of inflatable channels are in an inflated state.

3. The modular endovascular graft of claim 1 further comprising a stent secured to an end of the first graft body section and the first attachment element is disposed at an end of the first graft body section opposite the stent.

4. The modular endovascular graft of claim 3 further comprising a connector member disposed on the wall portions of the first and second graft body sections and the stent is secured to the connector member.

5. The modular endovascular graft of claim 1 wherein the first attachment element is at least partially secured to the second attachment element so as to form an axially overlapped portion having an axial length and wherein the axial length of the axially overlapped portion may vary depending on the relative axial position of the first graft body section and the second graft body section at the time when the attachment elements are secured to each other.

6. The modular endovascular graft of claim 1 wherein the first attachment element comprises a plurality of flexible hooks adjacent each other over a substantial area of the first wall portion and the second attachment element comprises a plurality of flexible loops adjacent each other over a substantial area of the second wall portion wherein the flexible hooks are configured to mechanically engage the flexible loops when the first attachment element is pressed against the second attachment element.

7. The modular endovascular graft of claim 1 wherein the first attachment element comprises a plurality of buttons having an enlarged head portion regularly spaced from each other on a surface of the first wall portion and the second attachment element comprises an expandable mesh having a plurality of apertures configured to allow entry of the enlarged head portion of the buttons while the mesh is in a circumferentially constrained state and to capture the enlarged head portion of the buttons when the mesh is in a circumferentially expanded state.

8. The modular endovascular graft of claim 1 wherein the first attachment element comprises a plurality of pins radially extending from a surface of the first wall portion and the second attachment element comprises an expandable mesh having a plurality of apertures configured to allow entry of the pins when the first attachment element is pressed against the second attachment element.

9. The modular endovascular graft of claim 1 wherein the inflatable cuff contains a curable material and the second attachment element comprises an expandable member with barbs configured to extend outwardly into the inflatable cuff and curable material.

10. The modular endovascular graft of claim 1 wherein the first attachment element comprises a plurality of protuberances radially extending from an outer surface of an expandable cylindrical member secured to the first graft body section and the second attachment element comprises an expandable mesh having a plurality of apertures configured to allow entry of the protuberances when the expandable cylindrical member is expanded and at least one protuberance is pressed into an opening of the expandable mesh of the second attachment element.

11. A modular endovascular graft, comprising:
a first graft body section having a first fluid flow lumen bounded by a first wall portion and a first attachment element that comprises a first inflatable element disposed on the first wall portion; and
a second graft body section having a second fluid flow lumen bounded by a second wall portion and a second attachment element disposed on the second wall portion which is configured to engage the first inflatable element when the first inflatable element is in an inflated state to prevent axial separation of the first and second graft body sections.

12. The modular endovascular graft of claim 11 wherein the first inflatable element comprises a first reduced circumference shoulder portion on an inner surface of the first wall portion of the first graft body section when the first inflatable element is in an inflated state, and
wherein the second attachment element comprises a second reduced circumference shoulder portion that is configured to mechanically engage the first reduced circumference shoulder portion to prevent axial separation of the first and second graft body sections.

13. The modular endovascular graft of claim 11 wherein the first graft body section further comprises a network of inflatable channels distributed over the first graft body section to provide structural rigidity and support to the first graft body section when the network of inflatable channels are in an inflated state.

14. The modular endovascular graft of claim 11 wherein the first graft body section comprises a plurality of first inflatable elements axially spaced along a longitudinal axis of the first graft body section from each other and the attachment element of the second graft body section is configured to engage any of the first inflatable elements when the first inflatable element is in an inflated state to allow adjustment of axial length of the joined graft body sections upon deployment.

15. The modular endovascular graft of claim 11 wherein the second graft body section comprises a plurality of second attachment elements axially spaced along a longitudinal axis of the second graft body section from each other and the inflatable element of the first graft body section is configured to engage any of the second attachment elements when the first inflatable element is in an inflated state to allow adjustment of axial length of the joined graft body sections upon deployment.

16. The modular endovascular graft of claim 11 wherein the inflatable element contains a curable material and the second attachment element comprises an expandable member with barbs configured to extend outwardly into the inflatable element and curable material.

17. The modular endovascular graft of claim 11 wherein the first graft body section further comprises a resilient member in the first wall portion axially adjacent the first inflatable element for enhanced engagement of the second attachment element.

18. The modular endovascular graft of claim 11 wherein the second attachment element comprises a recessed pocket configured to accept and engage the first inflatable element.

19. The modular graft of claim 11 wherein the second graft body section further comprises a tapered portion wherein the second fluid flow lumen tapers to an increased circumference in order to engage an inside surface of the first fluid flow lumen.

20. A method of treating a fluid vessel of a patient comprising:
providing a modular endovascular graft with a first graft body section having a first fluid flow lumen and a first inflatable element that comprises a first reduced circumference shoulder portion on an inner surface of the first graft body section when the element is in an inflated state and a second graft body section having a second fluid flow lumen and secured to the first graft body section by a second reduced circumference shoulder portion that mechanically engages the first reduced circumference shoulder portion to prevent axial separation of the first and second graft body sections;
deploying the first graft body section within a desired location of the patient's fluid flow vessel;
deploying the second graft body section adjacent the first graft body section;
positioning the second graft body section relative to the first graft body section such that the second attachment element is adjacent the first inflatable element; and
inflating the first inflatable element so as to engage the second attachment element and secure the first graft body section to the second graft body section.
